(19) United States
(12) Patent Application Publication
(10) Pub. No.: US 2009/0162876 A1
(43) Pub. Date: Jun. 25, 2009

(54) MYELOPEROXIDASE ASSAYS

(75) Inventors: Maciej Adamczyk, Gurnee, IL (US); Robert C. Doss, Libertyville, IL (US); Phillip G. Mattingly, Third Lake, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/201,097

(22) Filed: Aug. 29, 2008

Related U.S. Application Data
(60) Provisional application No. 61/015,449, filed on Dec. 20, 2007.

(51) Int. Cl. C12Q 1/28; G01N 33/543

(52) U.S. Cl. 435/7.92; 435/28; 435/7.1

(57) ABSTRACT

The present invention relates to methods and kits for determining autoantibodies to myeloperoxidase or a myeloperoxidase fragment and myeloperoxidase or a myeloperoxidase fragment in a test sample.

### RELATED APPLICATION INFORMATION

This application claims the priority of U.S. Provisional Application Ser. No. 61/015,449 (pending), the disclosure of which is incorporated herein by reference in its entirety.

### TECHNICAL FIELD

The present invention relates to assays and kits for detecting autoantibodies to myeloperoxidase or myeloperoxidase fragments in a test sample, determining the reliability of a myeloperoxidase assay result, and determining the amount of myeloperoxidase or myeloperoxidase fragments in a test sample.

### BACKGROUND

Cardiovascular disease (CVD) is the general term for heart and blood vessel diseases, including atherosclerosis, coronary heart disease, cerebrovascular disease and peripheral vascular disease. Cardiovascular disorders are acute manifestations of CVD and include myocardial infarction, stroke, angina pectoris, transient ischemic attacks and congestive heart failure. CVD accounts for one in every two deaths in the United States and is the number one killer disease. Thus, prevention of CVD is an area of major public health importance. Thereupon, early detection of CVD provides a greater opportunity for the initiation of treatment and the potential for recovery, especially in patients that are non-responsive to conventional therapy.

Cardiac markers or cardiac enzymes in the blood are often used in the diagnosis of CVD. These marker proteins are released into the bloodstream when damage to the heart occurs, such as, for example, in the case of a myocardial infarction. Examples of some well known marker proteins used in the diagnosis of CVD include, but are not limited to, troponin, brain natriuretic peptide (BNP), nt-proBNP, creatine kinase isoenzyme MB (CKMB), myoglobin, myeloperoxidase (MPO), choline, C-reactive protein (CRP), interleukin-6 (IL-6), tumor necrosis factor α (TNFα), placental growth factor (PlGF), Pregnancy-Associated Plasma Protein-A (PAPP-A), soluble CD40 (sCD40), and others.

As mentioned above, MPO is a marker protein used in the diagnosis of CVD. MPO (donor: hydrogen peroxide, oxidoreductase, EC 1.11.1.7) is a tetrameric, heavily glycosylated, basic (PI. 10) heme protein of approximately 150 kDa. It is comprised of two identical disulfide-linked protomers, each of which possesses a protoporphyrin-containing 59-64 kDa heavy subunit and a 14 kDa light subunit (See, Nauseef, W. M, et al., Blood, 67:1504-1507 (1986)). MPO is abundant in neutrophils and monocytes, accounting for 5% and 1 to 2%, respectively, of the dry weight of these cells (See, Nauseef, W. M, et al., Blood 67:1504-1507 (1986)). The heme protein is stored in primary azurophilic granules of leukocytes and secreted into both the extracellular milieu and the phagolysosomal compartment following phagocyte activation by a variety of agonists (See, Klebanoff, S. J, et al., The Neutrophil: Functions and Clinical Disorders. Amsterdam: Elsevier Scientific Publishing Co. (1978)).

A number of diagnostic tests for characterizing a subject's risk of developing or having CVD by assaying for MPO are known in the art. For example, U.S. Pat. No. 7,223,552 describes diagnostic tests that involve (1) determining the level of MPO activity in a test sample; (2) determining the level of MPO mass in a test sample; or (3) determining the level of MPO-generated oxidation products in a test sample. One of the problems with these diagnostic tests is the complication caused by the presence of autoantibodies in test samples. Specifically, the presence of autoantibodies in test samples have been observed to contribute to the generation of false negative results obtained in cardiac biomarker studies such as in troponin assays (See, for example, Bohner et al., Clin. Chem., 42, 2046 (1996)). Therefore, there is a need in the art for methods of detecting the presence of autoantibodies to MPO or MPO fragments in a test sample, methods for determining the reliability of an MPO assay result from a test sample, and methods for correctly determining the amount of MPO or MPO fragments in a test sample.

### SUMMARY

In one embodiment, the present invention relates to a method of determining the reliability of a myeloperoxidase assay result from a test sample. The method comprises the steps of:

(a) providing a test sample;

(b) providing a myeloperoxidase assay result;

(c) determining an amount of myeloperoxidase activity in the test sample;

(d) determining an amount of myeloperoxidase mass in the test sample; and

(e) comparing the amount of myeloperoxidase activity determined in step (c) with the amount of myeloperoxidase mass determined in step (d) and using said comparison to determine the reliability of the myeloperoxidase assay result provided in step (b).

In the above method, the determining of step (c) and the determining of step (d) are done simultaneously. Alternatively, the determining of step (c) and the determining of step (d) are done sequentially, in any order.

In the above method, the test sample is whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid or semen.

In the above method, the myeloperoxidase activity is determined using an immunoassay or a chemiluminescent assay. Additionally, in the above method, the myeloperoxidase mass is determined using an immunoassay or a chemiluminescent assay. Additionally, in the above method, the myeloperoxidase assay result is determined using an immunoassay or a chemiluminescent assay.

In another embodiment, the present invention relates to a method for detecting autoantibodies to myeloperoxidase or a myeloperoxidase fragment in a test sample. The method comprises the steps of:

(a) preparing a mixture comprising a test sample being assessed for autoantibodies to myeloperoxidase or a myeloperoxidase fragment and a first specific binding partner that is immobilized on a solid phase, wherein the first specific binding partner is myeloperoxidase or a myeloperoxidase fragment and further wherein the autoantibody and the first specific binding partner form a solid phase first specific binding partner-autoantibody complex;

(b) removing any unbound autoantibodies from the first specific binding partner-autoantibody complex;

(c) adding a second specific binding partner labeled with a detectable label to the mixture to form a first specific binding partner-autoantibody-second specific binding partner complex, wherein the second specific binding partner is an anti-human antibody and the detectable label is an acridinium compound;

(d) removing any unbound second specific binding partner labeled with a detectable label from the first specific binding partner-autoantibody-second specific binding partner complex;

(e) generating in or providing to the mixture a source of hydrogen peroxide before or after the addition of the second specific binding partner containing the detectable label;

(f) adding a basic solution to the mixture to generate a light signal; and

(g) measuring the light generated to detect the autoantibody.

In the above method, the test sample is whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid or semen. Moreover, in the above method, the hydrogen peroxide is provided by adding a buffer or a solution containing hydrogen peroxide. Alternatively, the hydrogen peroxide is generated by adding a hydrogen peroxide generating enzyme to the test sample.

The acridinium compound used in conjunction in the above method can be a acridinium-9-carboxamide. More specifically, the acridinium-9-carboxamide has a structure according to formula I:

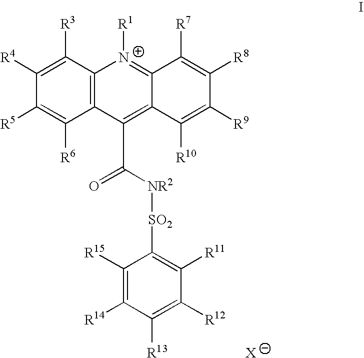

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{Θ} is an anion.

Alternatively, the acridinium compound used in the above method is an acridinium-9-carboxylate aryl ester. The acridinium-9-carboxylate aryl ester has a structure of formula II:

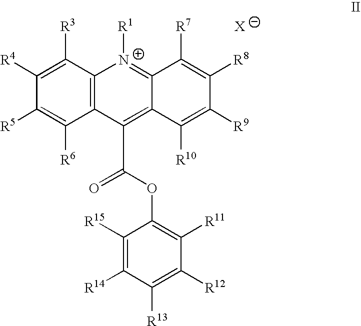

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{Θ} is an anion.

The above method can further comprise the step of quantifying the amount of autoantibodies to myeloperoxidase or myeloperoxidase fragment in the test sample by relating the amount of light generated in the test sample by comparison to a standard curve for said autoantibodies. The standard curve can be generated from solutions of autoantibodies of known concentrations.

In another embodiment, the present invention relates to an interdependent method for detecting autoantibodies to myeloperoxidase or a myeloperoxidase fragment and myeloperoxidase or a myeloperoxidase fragment in a test sample. The method comprises the steps of:

(a) adding a predetermined concentration of hydrogen peroxide to the test sample;

(b) adding an acridinium compound to the test sample before or after the addition of the hydrogen peroxide;

(c) adding a basic solution to the test sample to generate a light signal;

(d) measuring the light generated from the light signal and calculating the amount of myeloperoxidase or myeloperoxidase fragment present in the test sample; and

(e) performing a three dimensional dose response surface analysis to calculate the amount of autoantibodies in the test sample.

In the above method, the test sample is whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid or semen.

The above method can further comprise the step of quantifying the amount of myeloperoxidase or myeloperoxidase fragment in the test sample by relating the amount of light generated in the test sample by comparison to a standard curve for myeloperoxidase or myeloperoxidase fragment. Moreover, the standard curve can be generated from solutions of myeloperoxidase or myeloperoxidase fragment of a known concentration.

In the above method, the acridinium compound is an acridinium-9-carboxamide having a structure according to formula I:

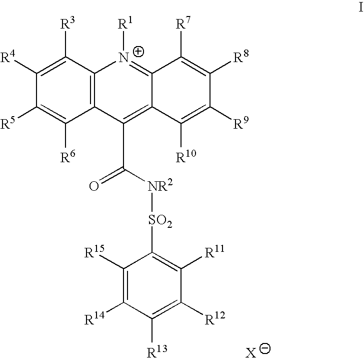

wherein R¹ and R² are each independently selected from the group consisting of: alkyl, alkenyl, alkynyl, aryl or aralkyl, sulfoalkyl and carboxyalkyl, and wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, and carboxyalkyl; and

optionally, if present, X^{Θ} is an anion.

Alternatively, the acridinium compound is an acridinium-9-carboxylate aryl ester having a structure according to formula II:

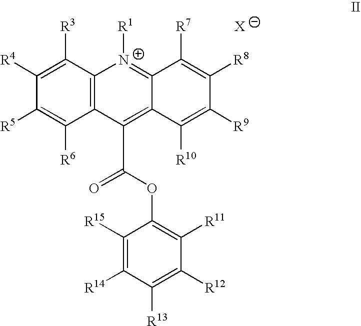

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{Θ} is an anion.

In another embodiment, the present invention relates to a method for determining the amount of myeloperoxidase or myeloperoxidase fragment in a test sample. The method comprises the steps of:

(a) determining an amount of myeloperoxidase activity in the test sample;

(b) determining an amount of myeloperoxidase mass in the test sample; and

(c) comparing the amount of myeloperoxidase activity determined in step (a) with the amount of myeloperoxidase mass determined in step (b) and using said comparison to determine the amount of myeloperoxidase or myeloperoxidase fragment in the test sample.

In the above method, the determining of step (a) and the determining of step (b) are done simultaneously. Alternatively, the determining of step (a) and the determining of step (b) are done sequentially, in any order.

In the above method, the test sample is whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid or semen. In the above method, the myeloperoxidase activity is determined using an immunoassay or a chemiluminescent assay. Additionally, in the above method, the myeloperoxidase mass is determined using an immunoassay or a chemiluminescent assay.

In another embodiment, the present invention relates to a test kit for detecting autoantibodies to myeloperoxidase or myeloperoxidase fragment in a test sample. The test kit comprises:

(a) a first specific binding partner, wherein said first specific binding partner is myeloperoxidase or a myeloperoxidase fragment; and

(b) a second specific binding partner, wherein said second specific binding partner is an anti-human antibody;

(c) at least one acridinium compound;

(d) at least one basic solution;

(e) a source of hydrogen peroxide; and

(f) instructions for detecting autoantibodies to myeloperoxidase or a myeloperoxidase fragment in a test sample.

The above test kit can further comprise a solid phase.

The acridinium compound in the test kit can be an acridinium-9-carboxamide having a structure according to formula I:

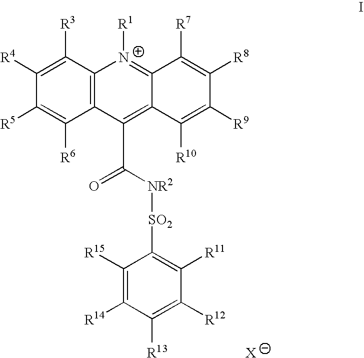

wherein R¹ and R² are each independently selected from the group consisting of: alkyl, alkenyl, alkynyl, aryl or aralkyl, sulfoalkyl and carboxyalkyl, and wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, and carboxyalkyl; and

optionally, if present, X^{Θ} is an anion.

Alternatively, the acridinium compound in the above test kit can be an acridinium-9-carboxylate aryl ester having a structure according to formula II:

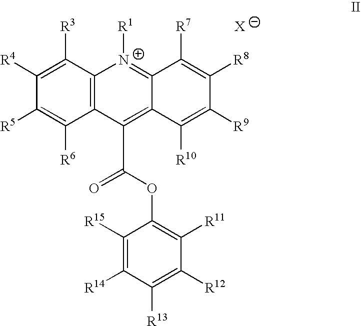

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{Θ} is an anion.

In another embodiment, the present invention relates to a test kit for detecting autoantibodies to myeloperoxidase or a myeloperoxidase fragment and myeloperoxidase or a myeloperoxidase fragment in a test sample. The kit comprises

(a) at least one acridinium compound;

(b) at least one basic solution;

(c) a source of hydrogen peroxide, wherein said source contains a predetermined amount of hydrogen peroxide; and

(d) instructions for performing a dimensional dose response surface analysis to calculate the amount of autoantibodies to myeloperoxidase or a myeloperoxidase fragment and myeloperoxidase or a myeloperoxidase fragment in the test sample.

In the above kit, the acridinium compound is an acridinium-9-carboxamide having a structure according to formula I:

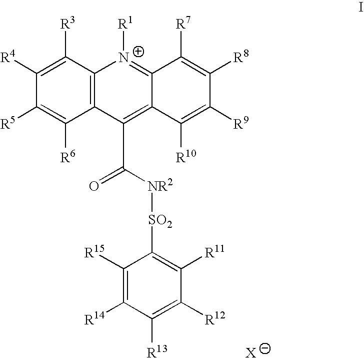

wherein R¹ and R² are each independently selected from the group consisting of: alkyl, alkenyl, alkynyl, aryl or aralkyl, sulfoalkyl and carboxyalkyl, and

wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, and carboxyalkyl; and

optionally, if present, X^{Θ} is an anion.

Alternatively in the above kit, the acridinium compound is an acridinium-9-carboxylate aryl ester having a structure according to formula II:

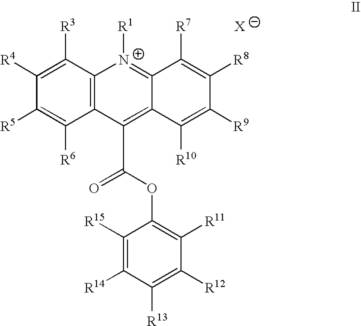

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{Θ} is an anion.

In yet another embodiment, the present invention relates to a test kit for determining the reliability of a myeloperoxidase assay result from a test sample. The kit comprises:

(a) one or more reagents for determining the amount of myeloperoxidase activity in the test sample;

(b) one or more reagents for determining the amount of myeloperoxidase mass in the test sample; and

(c) instructions for determining the reliability of a myeloperoxidase assay result from a test sample.

In yet another embodiment, the present invention relates to a test kit for determining the amount of myeloperoxidase or a myeloperoxidase fragment in a test sample. The kit comprises:

(a) one or more reagents for determining the amount of myeloperoxidase activity in the test sample;

(b) one or more reagents for determining the amount of myeloperoxidase mass in the test sample; and

(c) instructions for determining the amount of myeloperoxidase or myeloperoxidase fragment a test sample.

### DETAILED DESCRIPTION

The present invention relates to myeloperoxidase assays. The inventors have made the surprising discovery that the current methods of measuring the level of myeloperoxidase (MPO) or MPO fragment in a test sample may be inaccurate. In particular, current methods may underestimate the amount of physiologically relevant (i.e., that having physiological impact) MPO or MPO fragment in a test sample. A lower than actual MPO result may lead to incorrect diagnosis and/or treatment of a subject. For example, a patient presenting certain symptoms of a myocardial infection may not be appropriately treated in the critical early period if an MPO assay result is incorrectly below threshold levels.

While not wishing to be bound by any theory, a number of factors may lead to an artificially low MPO assay result. MPO mass assays are typically based on using antibodies specific for MPO or a MPO fragment in an enzyme-linked immunosorbent assay (ELISA). Such MPO mass assays may underestimate physiologically relevant MPO or MPO fragment due to modification of the MPO epitopes recognized by antibodies in the assay. For example, amino acid residues of the epitope may be chemically modified. In addition, epitopes may be removed MPO due to proteolytic processing.

Additionally, it is also known in the art that the presence of autoantibodies to MPO or MPO fragments in a test sample can contribute to the generation of false negative results obtained in MPO assays.

The present invention relates to a method of determining the reliability of a MPO assay result from a test sample. Methods for determining the reliability of a MPO assay result from a test sample are important in view of the fact that it is known in the art that the presence of autoantibodies to MPO or MPO fragments in a test sample can contribute to the generation of false negative results. The present invention also relates to a method for detecting autoantibodies to MPO or a MPO fragment in a test sample. The present invention further relates to methods for accurately and reliably determining the concentration or amount of MPO or MPO fragment in a test sample. Finally, the present invention also relates to kits for detecting autoantibodies to MPO or MPO fragments in a test sample, kits for determining the reliability of a MPO assay result from a test sample and test kits for accurately and reliably determining the concentration or amount of MPO or a MPO fragment in a test sample.

### A. DEFINITIONS

As used herein, the term “acyl” refers to a —C(O)Rₐ group where Rₐ is hydrogen, alkyl, cycloalkyl, cycloalkylalkyl, phenyl or phenylalkyl. Representative examples of acyl include, but are not limited to, formyl, acetyl, cylcohexylcarbonyl, cyclohexylmethylcarbonyl, benzoyl, benzylcarbonyl and the like.

As used herein, the term “alkenyl” means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

As used herein, the term “alkyl” means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

As used herein, the term “alkyl radical” means any of a series of univalent groups of the general formula CₙH₂ₙ₊₁ derived from straight or branched chain hydrocarbons.

As used herein, the term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

As used herein, the term “alkynyl” means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

As used herein, the term “amido” refers to an amino group attached to the parent molecular moiety through a carbonyl group (wherein the term “carbonyl group” refers to a —C(O)— group).

As used herein, the term “amino” means —NR_{b}R_{c}, wherein R_{b} and R_{c} are independently selected from the group consisting of hydrogen, alkyl and alkylcarbonyl.

As used herein, the term “angina pectoris” refers to chest discomfort caused by inadequate blood flow through the blood vessels (coronary vessels) of the myocardium.

As used herein, the term “analyte” refers to the substance to be detected, which may be suspected of being present in a sample (i.e., the test sample).

As used herein, the term “anion” refers to an anion of an inorganic or organic acid, such as, but not limited to, hydrochloric acid, hydrobromic acid, sulfuric acid, methane sulfonic acid, formic acid, acetic acid, oxalic acid, succinic acid, tartaric acid, mandelic acid, fumaric acid, lactic acid, citric acid, glutamic acid, aspartic acid, phosphate, trifluoromethansulfonic acid, trifluoroacetic acid and fluorosulfonic acid and any combinations thereof.

As used herein, the term “antibody” refers to an immunoglobulin molecule or immunologically active portion thereof, namely, an antigen-binding portion. Examples of immunologically active portions of immunoglobulin molecules include F(ab) and F(ab′)₂ fragments which can be generated by treating an antibody with an enzyme, such as pepsin. Examples of antibodies that can be used in the present invention include, but are not limited to, polyclonal antibodies, monoclonal antibodies, chimeric antibodies, human antibodies, humanized antibodies, recombinant antibodies, single-chain Fvs (“scFv”), an affinity maturated antibody, single chain antibodies, single domain antibodies, F(ab) fragments, F(ab′) fragments, disulfide-linked Fvs (“sdFv”), and antiidiotypic (“anti-Id”) antibodies and functionally active epitope-binding fragments of any of the above.

As used herein, the term “aralkyl” means an aryl group appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

As used herein, the term “aryl” means a phenyl group, or a bicyclic or tricyclic fused ring system wherein one or more of the fused rings is a phenyl group. Bicyclic fused ring systems are exemplified by a phenyl group fused to a cycloalkenyl group, a cycloalkyl group, or another phenyl group. Tricyclic fused ring systems are exemplified by a bicyclic fused ring system fused to a cycloalkenyl group, a cycloalkyl group, as defined herein or another phenyl group. Representative examples of aryl include, but are not limited to, anthracenyl, azulenyl, fluorenyl, indanyl, indenyl, naphthyl, phenyl, and tetrahydronaphthyl. The aryl groups of the present invention can be optionally substituted with one-, two, three, four, or five substituents independently selected from the group consisting of alkoxy, alkyl, carboxyl, halo, and hydroxyl.

As used herein, the terms “autoantibody” or “autoantibodies” refers an antibody or antibodies that binds to an analyte that is naturally occurring in the subject in which the antibody is produced. Preferably, the analyte is myeloperoxidase (MPO) or myeloperoxidase autoantibodies.

As used herein, the term “carboxy” or “carboxyl” refers to —CO₂H or —CO₂⁻.

As used herein, the term “carboxyalkyl” refers to a —(CH₂)ₙCO₂H or —(CH₂)ₙCO₂⁻ group where n is from 1 to 10.

As used herein, the term “cardiomyopathy” refers to a weakening of the heart muscle or a change in heart muscle structure. It is often associated with inadequate heart pumping or other heart function abnormalities. Cardiomyopathy can be caused by viral infections, heart attacks, alcoholism, long-term, severe high blood pressure, nutritional deficiencies (particularly selenium, thiamine, and L-camitine), systemic lupus erythematosus, celiac disease, and end-stage kidney disease. Types of cardiomyopathy include, but are not limited to, dilated cardiomyopathy, hypertrophic cardiomyopathy, and restrictive cardiomyopathy.

As used herein, the term “cyano” means a —CN group.

As used herein, the term “cycloalkenyl” refers to a non-aromatic cyclic or bicyclic ring system having from three to ten carbon atoms and one to three rings, wherein each five-membered ring has one double bond, each six-membered ring has one or two double bonds, each seven- and eight-membered ring has one to three double bonds, and each nine-to ten-membered ring has one to four double bonds. Representative examples of cycloalkenyl groups include cyclohexenyl, octahydronaphthalenyl, norbornylenyl, and the like. The cycloalkenyl groups can be optionally substituted with one, two, three, four, or five substituents independently selected from the group consisting of alkoxy, alkyl, carboxyl, halo, and hydroxyl.

As used herein, the term “cycloalkyl” refers to a saturated monocyclic, bicyclic, or tricyclic hydrocarbon ring system having three to twelve carbon atoms. Representative examples of cycloalkyl groups include cyclopropyl, cyclopentyl, bicyclo[3.1.1]heptyl, adamantyl, and the like. The cycloalkyl groups of the present invention can be optionally substituted with one, two, three, four, or five substituents independently selected from the group consisting of alkoxy, alkyl, carboxyl, halo, and hydroxyl.

As used herein, the term “cycloalkylalkyl” means a —R_{d}Rₑ group where R_{d} is an alkylene group and Rₑ is cycloalkyl group. A representative example of a cycloalkylalkyl group is cyclohexylmethyl and the like.

As used herein, the term “dilated cardiomyopathy” refers to a global, usually idiopathic, myocardial disorder characterized by a marked enlargement and inadequate function of the left ventricle. Dilated cardiomyopathy includes ischemic cardiomyopathy, idiopathic cardiomyopathy, hypertensive cardiomyopathy, infectious cardiomyopathy, alcoholic cardiomyopathy, toxic cardiomyopathy, and peripartum cardiomyopathy.

As used herein, the term “halogen” means a —Cl, —Br, —I or —F; the term “halide” means a binary compound, of which one part is a halogen atom and the other part is an element or radical that is less electronegative than the halogen, e.g., an alkyl radical.

As used herein, the term “hydrogen peroxide generating enzyme” refers to an enzyme that is capable of generating hydrogen peroxide. Examples of hydrogen peroxide generating enzymes are listed below in Table 1.

TABLE 1

| | IUBMB ENZYME | |
|---|---|---|
| ACCEPTED COMMON NAME | NOMENCLATURE | PREFERRED SUBSTRATE |
| (R)-6-hydroxynicotine oxidase | EC 1.5.3.6 | (R)-6-hydroxynicotine |
| (S)-2-hydroxy acid oxidase | EC 1.1.3.15 | S)-2-hydroxy acid |
| (S)-6-hydroxynicotine oxidase | EC 1.5.3.5 | (S)-6-hydroxynicotine |
| 3-aci-nitropropanoate oxidase | EC 1.7.3.5 | 3-aci-nitropropanoate |
| 3-hydroxyanthranilate oxidase | EC 1.10.3.5 | 3-hydroxyanthranilate |
| 4-hydroxymandelate oxidase | EC 1.1.3.19 | (S)-2-hydroxy-2-(4- |
| | | hydroxyphenyl)acetate |
| 6-hydroxynicotinate dehydrogenase | EC 1.17.3.3 | 6-hydroxynicotinate |
| Abscisic-aldehyde oxidase | EC 1.2.3.14 | abscisic aldehyde |
| acyl-CoA oxidase | EC 1.3.3.6 | acyl-CoA |
| Alcohol oxidase | EC 1.1.3.13 | a primary alcohol |
| aldehyde oxidase | EC 1.2.3.1 | an aldehyde |
| amine oxidase | | |
| amine oxidase (copper-containing) | EC 1.4.3.6 | primary monoamines, |
| | | diamines and histamine |
| amine oxidase (flavin-containing) | EC 1.4.3.4 | a primary amine |
| aryl-alcohol oxidase | EC 1.1.3.7 | an aromatic primary |
| | | alcohol |
| | | (2-naphthyl)methanol |
| | | 3-methoxybenzyl alcohol |
| aryl-aldehyde oxidase | EC 1.2.3.9 | an aromatic aldehyde |
| catechol oxidase | EC 1.1.3.14 | Catechol |
| cholesterol oxidase | EC 1.1.3.6 | Cholesterol |
| choline oxidase | EC 1.1.3.17 | Choline |
| columbamine oxidase | EC 1.21.3.2 | Columbamine |
| cyclohexylamine oxidase | EC 1.4.3.12 | Cyclohexylamine |
| cytochrome c oxidase | EC 1.9.3.1 | |
| D-amino-acid oxidase | EC 1.4.3.3 | a D-amino acid |
| D-arabinono-1,4-lactone oxidase | EC 1.1.3.37 | D-arabinono-1,4-lactone |
| D-arabinono-1,4-lactone oxidase | EC 1.1.3.37 | D-arabinono-1,4-lactone |
| D-aspartate oxidase | EC 1.4.3.1 | D-aspartate |
| D-glutamate oxidase | EC 1.4.3.7 | D-glutamate |
| D-glutamate(D-aspartate) oxidase | EC 1.4.3.15 | D-glutamate |
| dihydrobenzophenanthridine oxidase | EC 1.5.3.12 | dihydrosanguinarine |
| dihydroorotate oxidase | EC 1.3.3.1 | (S)-dihydroorotate |
| dihydrouracil oxidase | EC 1.3.3.7 | 5,6-dihydrouracil |
| dimethylglycine oxidase | EC 1.5.3.10 | N,N-dimethylglycine |
| D-mannitol oxidase | EC 1.1.3.40 | Mannitol |
| ecdysone oxidase | EC 1.1.3.16 | Ecdysone |
| ethanolamine oxidase | EC 1.4.3.8 | Ethanolamine |
| galactose oxidase | EC 1.1.3.9 | D-galactose |
| glucose oxidase | EC 1.1.3.4 | β-D-glucose |
| glutathione oxidase | EC 1.8.3.3 | Glutathione |
| glycerol-3-phosphate oxidase | EC 1.1.3.21 | sn-glycerol 3-phosphate |
| glycine oxidase | EC 1.4.3.19 | Glycine |
| glyoxylate oxidase | EC 1.2.3.5 | Glyoxylate |
| hexose oxidase | EC 1.1.3.5 | D-glucose, |
| | | D-galactose |
| | | D-mannose |
| | | maltose |
| | | lactose |
| | | cellobiose |
| hydroxyphytanate oxidase | EC 1.1.3.27 | L-2-hydroxyphytanate |
| indole-3-acetaldehyde oxidase | EC 1.2.3.7 | (indol-3-yl)acetaldehyde |
| lactic acid oxidase | | Lactic acid |
| L-amino-acid oxidase | EC 1.4.3.2 | an L-amino acid |
| L-aspartate oxidase | EC 1.4.3.16 | L-aspartate |
| L-galactonolactone oxidase | EC 1.3.3.12 | L-galactono-1,4-lactone |
| L-glutamate oxidase | EC 1.4.3.11 | L-glutamate |
| L-gulonolactone oxidase | EC 1.1.3.8 | L-gulono-1,4-lactone |
| L-lysine 6-oxidase | EC 1.4.3.20 | L-lysine |
| L-lysine oxidase | EC 1.4.3.14 | L-lysine |
| long-chain-alcohol oxidase | EC 1.1.3.20 | A long-chain-alcohol |
| L-pipecolate oxidase | EC 1.5.3.7 | L-pipecolate |
| L-sorbose oxidase | EC 1.1.3.11 | L-sorbose |
| malate oxidase | EC 1.1.3.3 | (S)-malate |
| methanethiol oxidase | EC 1.8.3.4 | Methanethiol |
| monoamino acid oxidase | | |
| N⁶-methyl-lysine oxidase | EC 1.5.3.4 | 6-N-methyl-L-lysine |
| N-acylhexosamine oxidase | EC 1.1.3.29 | N-acetyl-D-glucosamine |
| | | N-glycolylglucosamine |
| | | N-acetylgalactosamine |
| | | N-acetylmannosamine. |
| NAD(P)H oxidase | EC 1.6.3.1 | NAD(P)H |
| nitroalkane oxidase | EC 1.7.3.1 | a nitroalkane |
| N-methyl-L-amino-acid oxidase | EC 1.5.3.2 | an N-methyl-L-amino |
| | | acid |
| nucleoside oxidase | EC 1.1.3.39 | Adenosine |
| oxalate oxidase | EC 1.2.3.4 | Oxalate |
| polyamine oxidase | EC 1.5.3.11 | 1-N-acetylspermine |
| polyphenol oxidase | EC 1.14.18.1 | |
| polyvinyl-alcohol oxidase | EC 1.1.3.30 | polyvinyl alcohol |
| prenylcysteine oxidase | EC 1.8.3.5 | an S-prenyl-L-cysteine |
| protein-lysine 6-oxidase | EC 1.4.3.13 | peptidyl-L-lysyl-peptide |
| putrescine oxidase | EC 1.4.3.10 | butane-1,4-diamine |
| pyranose oxidase | EC 1.1.3.10 | D-glucose |
| | | D-xylose |
| | | L-sorbose |
| | | D-glucono-1,5-lactone |
| pyridoxal 5′-phosphate synthase | EC 1.4.3.5 | pyridoxamine 5′- |
| | | phosphate |
| pyridoxine 4-oxidase | EC 1.1.3.12 | Pyridoxine |
| pyrroloquinoline-quinone synthase | EC 1.3.3.11 | 6-(2-amino-2- |
| | | carboxyethyl)-7,8-dioxo- |
| | | 1,2,3,4,5,6,7,8- |
| | | octahydroquinoline-2,4- |
| | | dicarboxylate |
| pyruvate oxidase | EC 1.2.3.3 | Pyruvate |
| pyruvate oxidase (CoA-acetylating) | EC 1.2.3.6 | Pyruvate |
| reticuline oxidase | EC 1.21.3.3 | Reticuline |
| retinal oxidase | EC 1.2.3.11 | Retinal |
| rifamycin-B oxidase | EC 1.10.3.6 | rifamycin-B |
| sarcosine oxidase | EC 1.5.3.1 | Sarcosine |
| secondary-alcohol oxidase | EC 1.1.3.18 | a secondary alcohol |
| sulfite oxidase | EC 1.8.3.1 | Sulfite |
| superoxide dismutase | EC 1.15.1.1 | Superoxide |
| superoxide reductase | EC 1.15.1.2 | Superoxide |
| tetrahydroberberine oxidase | EC 1.3.3.8 | (S)-tetrahydroberberine |
| thiamine oxidase | EC 1.1.3.23 | Thiamine |
| tryptophan α,β-oxidase | EC 1.3.3.10 | L-tryptophan |
| urate oxidase (uricase, uric acid | EC 1.7.3.3 | uric acid |
| oxidase) | | |
| vanillyl-alcohol oxidase | EC 1.1.3.38 | vanillyl alcohol |
| xanthine oxidase | EC 1.17.3.2 | Xanthine |
| xylitol oxidase | EC 1.1.3.41 | Xylitol |

As used herein, the term “hydroxyl” means an —OH group.

As used herein, the term “hypertrophic cardiomyopathy” refers to a condition resulting from the right and left heart muscles growing to be different sizes.

As used herein, the term “ischemic heart disease” refers to any condition in which heart muscle is damaged or works inefficiently because of an absence or relative deficiency of its blood supply; most often caused by atherosclerosis, it includes angina pectoris, acute myocardial infarction, and chronic ischemic heart disease.

As used herein, the term “myeloperoxidase activity” refers to the turnover or consumption of a substrate based on a quantifiable amount (e.g., mass) of a myeloperoxidase or a myeloperoxidase fragment. In other words, myeloperoxidase activity refers to the amount of myeloperoxidase or myeloperoxidase fragment needed to convert or change a substrate into the requisite product in a given time. Methods for determining or quantifying myeloperoxidase activity are well known in the art. For example, one method that could be used to determine myeloperoxidase activity is an immunoassay (such as, for example, affinity chromatography, immunoelectrophoresis, radioimmunoassay (RIA), enzyme-linked immunosorbent assays (ELISAs), immunofluorescent assays, Western blotting, and the like). Such immunoassays can be homogeneous or heterogeneous immunoassays. Alternatively, myeloperoxidase activity can be determined using a chemiluminescent assay such as that described in U.S. application Ser. No. 11/842,897 filed on Aug. 21, 2007 entitled, “Measurement of Haloperoxidase Activity With Chemiluminescent Detection”, the contents of which are herein incorporated by reference. Still another method that can be used to determine myeloperoxidase activity is a colorimetric-based assay where a chromophore that serves as a substrate for the peroxidase generates a product with a characteristic wavelength which may be followed by any of various spectroscopic methods including UV-visible or fluorescence detection such as that described in U.S. Pat. No. 7,223,552, the contents of which are also incorporated by reference in their entirety.

As used herein, the term “myeloperoxidase fragment” or “MPO fragment” refers to a peptide or protein that comprise fewer amino acids than the full-length human myeloperoxidase.

As used herein, the term “myeloperoxidase mass” refers to the mass or weight of myeloperoxidase or a myeloperoxidase fragment. The mass of myeloperoxidase or a myeloperoxidase fragment in a given test sample can be determined using routine techniques known in the art. For example, the mass of myeloperoxidase or a myeloperoxidase fragment in a test sample can be determined using an immunoassay (such as, for example, affinity chromatography, immunoelectrophoresis, radioimmunoassay (RIA), enzyme-linked immunosorbent assays (ELISAs), immunofluorescent assays, Western blotting, and the like). Such immunoassays can be homogeneous or heterogeneous immunoassays. For example, the myeloperoxidase ELISA Kit commercially available from Calbiochem® (Catalog No. 475919) can be used. Alternatively, the mass of myeloperoxidase can be determined using a chemiluminescent assay such as that described in U.S. application Ser. No. 11/842,897 filed on Aug. 21, 2007 entitled, “Measurement of Haloperoxidase Activity With Chemiluminescent Detection”, the contents of which are herein incorporated by reference. Still another method that can be used to determine the mass of myeloperoxidase is in situ peroxidase staining of the bodily sample as described in U.S. Pat. No. 7,223,552, the contents of which are herein incorporated by reference.

As used herein, the term “myocardial infarction” (heart attack) refers to when an area of heart muscle dies or is damaged because of an inadequate supply of oxygen to that area.

As used herein, the term “myocarditis” refers to inflammation of the myocardium. Myocarditis can be caused by a variety of conditions such as viral infection, sarcoidosis, rheumatic fever, autoimmune diseases (such as systemic lupus erythematosus, etc.), and pregnancy.

As used herein, the term “nitro” means a —NO₂ group.

As used herein, the term “oxoalkyl” refers to —(CH₂)ₙC(O)Rₐ, where Rₐ is hydrogen, alkyl, cycloalkyl, cycloalkylalkyl, phenyl or phenylalkyl and where n is from 1 to 10.

As used herein, the term “phenylalkyl” means an alkyl group which is substituted by a phenyl group.

As used herein, the term “reliability” means that with respect to a given result or value (such as that obtained from an assay, such as an immunoassay or a chemiluminescent assay) that there is at least about a 90% certainty (e.g., from about a 90% certainty to about a 100% certainty) that said result or value is accurate or correct, preferably at least about a 95% certainty (e.g., from about a 95% certainty to about a 100% certainty) that said result or value is accurate or correct.

As used herein, the term “restrictive cardiomyopathy” refers to a condition characterized by the heart muscle's inability to relax between contractions, which prevents it from filling sufficiently.

As used herein, the term “risk” refers to the possibility or probability of a particular event occurring either presently, or, at some point in the future. “Risk stratification” refers to an arraying of known clinical risk factors to allow physicians to classify patients into a low, moderate, high or highest risk of developing of a particular disease, disorder or condition.

As used herein, the phrase “specific binding partner,” as used herein, is a member of a specific binding pair. That is, two different molecules where one of the molecules, through chemical or physical means, specifically binds to the second molecule. Therefore, in addition to antigen and antibody specific binding pairs of common immunoassays, other specific binding pairs can include biotin and avidin, carbohydrates and lectins, complementary nucleotide sequences, effector and receptor molecules, cofactors and enzymes, enzyme inhibitors, and enzymes and the like. Furthermore, specific binding pairs can include members that are analogs of the original specific binding members, for example, an analyte-analog. Immunoreactive specific binding members include antigens, antigen fragments, antibodies and antibody fragments, both monoclonal and polyclonal and complexes thereof, including those formed by recombinant DNA molecules.

As used herein, the term “sulfo” means a —SO₃H group.

As used herein, the term “sulfoalkyl” refers to a —(CH₂)ₙSO₃H or —(CH₂)ₙSO₃⁻ group where n is from 1 to 10.

As used herein, the term “test sample” generally refers to a biological material being tested for and/or suspected of containing an analyte of interest, such as a myeloperoxidase. The test sample may be derived from any biological source, such as, a physiological fluid, including, but not limited to, whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen and so forth. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

As used in this specification and the appended claims, the singular forms “a”, “an”, and “the” include plural references unless the context clearly dictates otherwise. Thus, for example, a reference to “the method” includes one or more methods, and/or steps of the type described herein and/or which will become apparent to those persons skilled in the art upon reading this disclosure and so forth.

### B. METHODS FOR DETERMINING THE RELIABILITY OF A MPO ASSAY RESULT FROM A TEST SAMPLE

In one embodiment, the present invention relates to a method of determining the reliability of a MPO assay result from a test sample. Specifically, the methods of the present invention allow one to determine whether or not a result or value obtained in an assay for MPO or a MPO fragment, such as, but not limited to, an immunoassay or a chemiluminescent assay, is reliable or correct or whether said result or value in not reliable or correct but may instead be a false negative result.

The assay or method of the present invention involves obtaining a test sample from a subject. A subject from which a test sample can be obtained is any vertebrate. Preferably, the vertebrate is a mammal. Examples of mammals include, but are not limited to, dogs, cats, rabbits, mice, rats, goats, sheep, cows, pigs, horses, non-human primates and humans. The test sample can be obtained from the subject using routine techniques known to those skilled in the art. Preferably, the test sample contains MPO or a MPO fragment.

The method involves determining an amount of MPO activity in a test sample and the amount of MPO mass in the test sample. The determination of the amount of MPO activity in the test sample the amount of MPO mass in the test sample can be performed simultaneously or sequentially, in any order.

The method also involves obtaining the amount of MPO or MPO fragment (namely, MPO mass) in the test sample obtained from an assay (“MPO assay result”). The MPO assay result can be obtained using any assay known in the art. For example, the MPO assay result can be obtained from an immunoassay, a chemiluminescent assay, etc. The MPO assay result from the test sample can be obtained at any time during the performance of the method. For example, the MPO assay result can be obtained before the MPO activity in the test sample is determined, after the MPO activity in the test sample is determined, before the MPO mass in the test sample is determined, after the MPO mass in the test sample is determined, after the MPO activity in the test sample is determined but prior to when the MPO mass in the test sample is determined, after the MPO mass in the test sample is determined but before the MPO activity in the test sample is determined, before the MPO activity in the test sample is determined and the MPO mass in the test sample is determined, after both the MPO activity in the test sample and MPO mass in the test sample is determined, simultaneously with the determination of the MPO activity in the test sample, simultaneously with the determination of the MPO mass in the test sample or simultaneously with the determination of the MPO activity in the test sample and MPO mass in the test sample.

After the amount of MPO activity in the test sample and the amount of MPO mass in the test sample have been determined, then a comparison of these values is performed. The manner in which the comparison is made is not critical. For example, the values for each the amount of MPO activity and the amount of MPO mass can be expressed as a ratio. The comparison can be made manually, such as by a human, or can completely or partially be performed by a computer program or algorithm, along with the necessary hardware, such as input, memory, processing display and output devices.

The value obtained for the amount of MPO activity in the test sample and the value obtained for the amount of MPO mass in the test sample should be equal (or if expressed as a ratio should be in the ratio of about 1:1) or if not equal, should not vary by more than about ten percent (10%; e.g., from about 0.1% to about 10%), preferably, not more than about five percent (5%;; e.g., from about 0.1% to about 5%).

After the above described comparison is made between the values for MPO activity and MPO mass, a determination is made as to the correctness or reliability of the MPO assay result. Specifically, if the result of the above comparison is that the value obtained for MPO activity and the value of MPO mass are equal or, if not equal, vary less than by about ten percent (10%; e.g., from about 0.1% to about 10%), then a conclusion can be made that the MPO assay result is correct or reliable. However, if the result of the above comparison is that the value obtained for MPO activity and the value of MPO mass are not equal and vary more than by about ten percent (10%; e.g., from about 10.1% to about 100%), then a conclusion can be made that the MPO assay result is not correct or reliable.

### C. METHODS FOR DETECTING AUTOANTIBODIES TO MPO OR A MPO FRAGMENT IN A TEST SAMPLE

In another embodiment, the present invention relates to methods for detecting autoantibodies to MPO or a MPO fragment in a test sample. As discussed previously herein, the presence of autoantibodies to MPO or a MPO fragment in a test sample can contribute to the generation of false negative results obtained in MPO assays. Therefore, the methods of the present invention allow one to learn prior to performing a MPO assay whether or not a test sample might contain autoantibodies to MPO or a MPO fragment that might contribute to the generation of a false negative. Alternatively, the methods of the present invention provide one with a means necessary to confirm or question the correctness or reliability of a MPO assay result.

The assay or method of the present invention involves obtaining a test sample from a subject. A subject from which a test sample can be obtained is any vertebrate. Preferably, the vertebrate is a mammal. Examples of mammals include, but are not limited to, dogs, cats, rabbits, mice, rats, goats, sheep, cows, pigs, horses, non-human primates and humans. The test sample can be obtained from the subject using routine techniques known to those skilled in the art. Preferably, the test sample contains MPO or a MPO fragment.

In one aspect, after the test sample is obtained from a subject, a first mixture is prepared. The mixture contains the test sample being assessed for autoantibodies to MPO or a MPO fragment and a first specific binding partner, wherein the first specific binding partner and any autoantibodies contained in the test sample form a first specific binding partner-autoantibody complex. Preferably, the first specific binding partner is MPO or a fragment of MPO. The order in which the test sample and first specific binding partner are added to form the mixture is not critical. Preferably, the first specific binding partner is immobilized on a solid phase. The solid phase used in the immunoassay (for the first specific binding partner and optionally, the second specific binding partner) can be any solid phase known in the art, such as, but not limited to, a magnetic particle, bead, test tube, microtiter plate, cuvette, membrane, a scaffolding molecule, film, filter paper, disc and chip.

After the mixture containing the first specific binding partner-autoantibody complex is formed, any unbound autoantibodies are removed from said complex using any technique known in the art, such as washing.

After any unbound autoantibodies are removed, a second specific binding partner is added to the mixture to form a first specific binding partner-autoantibody-second specific binding partner complex. The second specific binding partner is preferably an anti-human antibody. Moreover, also preferably, the second specific binding partner is labeled with or contains a detectable label. In terms of the detectable label, any detectable label known in the art can be used. For example, the detectable label can be a radioactive label (such as, e.g., ³H, ¹²⁵I, ³⁵S, ¹⁴C, ³²P, and ³³P), an enzymatic label (such as, e.g., horseradish peroxidase, alkaline peroxidase, glucose 6-phosphate dehydrogenase, and the like), a chemiluminescent label (such as, e.g., acridinium esters, luminal, isoluminol, thioesters, sulfonamides, phenanthridinium esters, and the like), a fluorescence label (such as, e.g., fluorescein (e.g., 5-fluorescein, 6-carboxyfluorescein, 3′6-carboxyfluorescein, 5(6)-carboxyfluorescein, 6-hexachlorofluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997) and in Haugland, Handbook of Fluorescent Probes and Research Chemicals (1996), which is a combined handbook and catalogue published by Molecular Probes, Inc., Eugene, Oreg. Preferably, however, the detectable label is an acridinium compound that can be used in a chemiluminescent assay. Preferably, the acridinium compound is an acridinium-9-carboxamide. Specifically, the acridinium-9-carboxamide has a structure according to formula I:

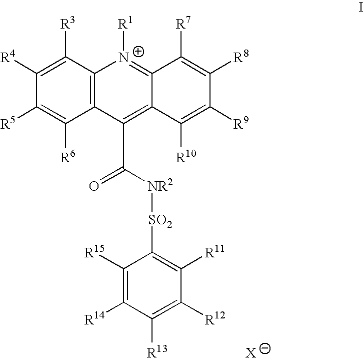

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl;

and further wherein any of the alkyl, alkenyl, alkynyl, aryl or aralkyl may contain one or more heteroatoms; and

optionally, if present, X^{Θ} is an anion.

Methods for preparing acridinium 9-carboxamides are described in Mattingly, P. G. J. Biolumin. Chemilumin., 6, 107-14; (1991); Adamczyk, M.; Chen, Y.-Y., Mattingly, P. G.; Pan, Y. J. Org. Chem., 63, 5636-5639 (1998); Adamczyk, M.; Chen, Y.-Y.; Mattingly, P. G.; Moore, J. A.; Shreder, K. Tetrahedron, 55, 10899-10914 (1999); Adamczyk, M.; Mattingly, P. G.; Moore, J. A.; Pan, Y. Org. Lett., 1, 779-781 (1999); Adamczyk, M.; Chen, Y.-Y.; Fishpaugh, J. R.; Mattingly, P. G.; Pan, Y.; Shreder, K.; Yu, Z. Bioconjugate Chem., 11, 714-724 (2000); Mattingly, P. G.; Adamczyk, M. In Luminescence Biotechnology: Instruments and Applications; Dyke, K. V. Ed.; CRC Press: Boca Raton, pp. 77-105 (2002); Adamczyk, M.; Mattingly, P. G.; Moore, J. A.; Pan, Y. Org. Lett., 5, 3779-3782 (2003); and U.S. Pat. Nos. 5,468,646, 5,543,524 and 5,783,699 (each incorporated herein by reference in their entireties for their teachings regarding same).

Alternatively, the acridinium compound can be an acridinium-9-carboxylate aryl ester; the acridinium-9-carboxylate aryl ester can have a structure according to formula II:

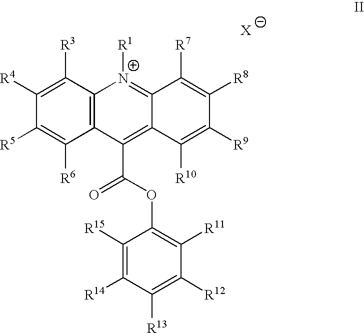

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{Θ} is an anion.

Examples of acridinium-9-carboxylate aryl esters having the above formula II that can be used in the present invention include, but are not limited to, 10-methyl-9-(phenoxycarbonyl)acridinium fluorosulfonate (available from Cayman Chemical, Ann Arbor, Mich.). Methods for preparing acridinium 9-carboxylate aryl esters are described in McCapra, F., et al., Photochem. Photobiol., 4, 1111-21 (1965); Razavi, Z et al., Luminescence, 15:245-249 (2000); Razavi, Z et al., Luminescence, 15:239-244 (2000); and U.S. Pat. No. 5,241,070 (each incorporated herein by reference in their entireties for their teachings regarding same).

After the addition of the second specific binding partner and after the formation of the first specific binding partner-autoantibody-second specific binding complex, any unbound second specific binding partner (whether labeled or unlabeled) is removed from said complex using any technique known in the art, such as washing.

In one embodiment of the present invention, hydrogen peroxide is generated in situ in the mixture or provided or supplied to the mixture before the addition of the above-described acridinium compound (specifically, the second specific binding partner labeled with the acridinium compound). In a second embodiment of the present invention, the hydrogen peroxide is generated in situ in the mixture or provided or supplied to the mixture simultaneously with the above-described acridinium compound (specifically, the second specific binding partner labeled with the acridinium compound). In a third embodiment, hydrogen peroxide is generated in situ or provided or supplied to the mixture after the above-described acridinium compound (specifically, the second specific binding partner labeled with the acridinium compound) is added to the test sample.

As mentioned above, hydrogen peroxide can be generated in situ in the mixture. Hydrogen peroxide can be generated in situ in a number of ways. For example, a hydrogen peroxide generating enzyme can be added to the first mixture. Specifically, one or more hydrogen peroxide generating enzymes can be added to the mixture in an amount sufficient to allow for the generation of hydrogen peroxide in situ in the mixture. The amount of one or more of the above enzymes to be added to the mixture can be readily determined by one skilled in the art.

Hydrogen peroxide can also be generated electrochemically in situ as shown in Agladze, G. R.; Tsurtsumia, G. S.; Jung, B. I.; Kim, J. S.; Gorelishvili, G. J. Applied Electrochem., 37, 375-383 (2007); Qiang, Z.; Chang, J.-H.; Huang, C.-P. Water Research, 36, 85-94 (2002), for example. Hydrogen peroxide can also be generated photochemically in situ, e.g. Draper, W. M.; Crosby, D. G. Archives of Environmental Contamination and Toxicology, 12, 121-126 (1983).

Alternatively, a source of hydrogen peroxide can be supplied to or provided in the mixture. For example, the source of the hydrogen peroxide can be one or more buffers or other solutions that are known to contain hydrogen peroxide. Such buffers or other solutions are simply added to the mixture. Alternatively, another source of hydrogen peroxide can simply be a solution containing hydrogen peroxide.

As demonstrated by the above, the timing and order in which the acridinium compound (specifically, the second specific binding partner labeled with the acridinium compound) and the hydrogen peroxide provided in or supplied to or generated in situ in the mixture is not critical provided that they are added, provided, supplied or generated in situ prior to the addition of at least one basic solution, which will be discussed in more detail below.

After the addition of the acridinium compound (specifically, the second specific binding partner labeled with the acridinium compound) and the hydrogen peroxide to the mixture, at least one basic solution is added to the mixture in order to generate a detectable signal, namely, a chemiluminescent signal. The basic solution is a solution that contains at least one base and that has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate and calcium bicarbonate. The amount of basic solution added to the mixture depends on the concentration of the basic solution used in the assay. Based on the concentration of the basic solution used, one skilled in the art could easily determine the amount of basic solution to be used in the method. Chemiluminescent signals generated can be detected using routine techniques known to those skilled in the art.

Thus, in the assay of the present invention, the chemiluminescent signal generated after the addition of a basic solution indicates the presence in the test sample of autoantibodies to MPO to a MPO fragment. The amount of the autoantibodies in the test sample can be quantified based on the intensity of the signal generated. Specifically, the amount of autoantibodies contained in a test sample is inversely proportional to the intensity of the signal generated. Specifically, the amount of autoantibodies present can be quantified based on comparing the amount of light generated to a standard curve for autoantibodies to MPO or a MPO fragment or by comparison to a reference standard. The standard curve can be generated using serial dilutions or solutions of the autoantibodies to MPO or a MPO fragment of known concentration, by mass spectroscopy, gravimetrically and by other techniques known in the art.

In another aspect, the present invention relates to an interdependent method of detecting autoantibodies to MPO or a MPO fragment as well as MPO or a MPO fragment in a test sample. The assay or method of the present invention involves obtaining a test sample from a subject. A subject from which a test sample can be obtained is any vertebrate. Preferably, the vertebrate is a mammal. Examples of mammals include, but are not limited to, dogs, cats, rabbits, mice, rats, goats, sheep, cows, pigs, horses, non-human primates and humans. The test sample can be obtained from the subject using routine techniques known to those skilled in the art. Preferably, the test sample contains MPO or a MPO fragment.

The method also involves adding a predetermined (or known) concentration (or amount) of hydrogen peroxide to a test sample. The hydrogen peroxide to be added to the test sample can be in the form of one or more buffers or other solutions that are known to contain hydrogen peroxide. Such buffers or other solutions are simply added to the test sample. Alternatively, the hydrogen peroxide can simply be a solution containing hydrogen peroxide. The predetermined concentration or amount of hydrogen peroxide to be added to the test sample can be readily determined by one skilled in the art.

In the above method, an acridinium compound is added to the test sample. Preferably, the acridinium compound is an acridinium-9-carboxamide. Specifically, the acridinium-9-carboxamide has a structure according to formula I:

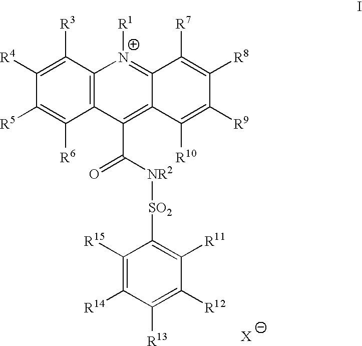

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl;

and further wherein any of the alkyl, alkenyl, alkynyl, aryl or aralkyl may contain one or more heteroatoms; and

optionally, if present, X^{Θ} is an anion.

Methods for preparing acridinium 9-carboxamides are described in Mattingly, P. G. J. Biolumin. Chemilumin., 6, 107-14; (1991); Adamczyk, M.; Chen, Y.-Y., Mattingly, P. G.; Pan, Y. J. Org. Chem., 63, 5636-5639 (1998); Adamczyk, M.; Chen, Y.-Y.; Mattingly, P. G.; Moore, J. A.; Shreder, K. Tetrahedron, 55, 10899-10914 (1999); Adamczyk, M.; Mattingly, P. G.; Moore, J. A.; Pan, Y. Org. Lett., 1, 779-781 (1999); Adamczyk, M.; Chen, Y.-Y.; Fishpaugh, J. R.; Mattingly, P. G.; Pan, Y.; Shreder, K.; Yu, Z. Bioconjugate Chem., 11, 714-724 (2000); Mattingly, P. G.; Adamczyk, M. In Luminescence Biotechnology: Instruments and Applications; Dyke, K. V. Ed.; CRC Press: Boca Raton, pp. 77-105 (2002); Adamczyk, M.; Mattingly, P. G.; Moore, J. A.; Pan, Y. Org. Lett., 5, 3779-3782 (2003); and U.S. Pat. Nos. 5,468,646, 5,543,524 and 5,783,699 (each incorporated herein by reference in their entireties for their teachings regarding same).

Alternatively, the acridinium compound can be an acridinium-9-carboxylate aryl ester; the acridinium-9-carboxylate aryl ester can have a structure according to formula II:

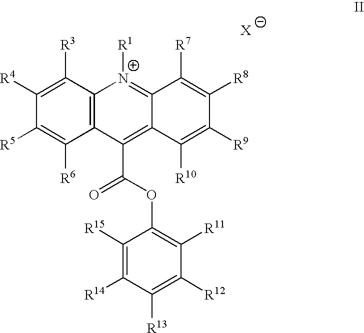

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{Θ} is an anion.

Examples of acridinium-9-carboxylate aryl esters having the above formula II that can be used in the present invention include, but are not limited to, 10-methyl-9-(phenoxycarbonyl)acridinium fluorosulfonate (available from Cayman Chemical, Ann Arbor, Mich.). Methods for preparing acridinium 9-carboxylate aryl esters are described in McCapra, F., et al., Photochem. Photobiol., 4, 1111-21 (1965); Razavi, Z et al., Luminescence, 15:245-249 (2000); Razavi, Z et al., Luminescence, 15:239-244 (2000); and U.S. Pat. No. 5,241,070 (each incorporated herein by reference in their entireties for their teachings regarding same).

The timing and order in which the acridinium compound and the hydrogen peroxide is supplied to the test sample is not critical provided that each of the acridinium compound and the hydrogen peroxide is added to the test sample prior to the addition of at least one basic solution, which will be discussed in more detail below.

After the addition of the acridinium compound and the hydrogen peroxide to the test sample, at least one basic solution is added to the test sample in order to generate a detectable signal, namely, a first chemiluminescent signal. The basic solution is a solution that contains at least one base and that has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate and calcium bicarbonate. The amount of basic solution added to the test sample depends on the concentration of the basic solution used in the assay. Based on the concentration of the basic solution used, one skilled in the art could easily determine the amount of basic solution to be used in the method. Chemiluminescent signals generated can be detected using routine techniques known to those skilled in the art.

Thus, the chemiluminescent signal generated after the addition of a basic solution, indicates the presence of MPO or a MPO fragment. The amount of MPO or MPO fragment (namely, the MPO or MPO fragment mass) in the test sample can be quantified based on the intensity of the first signal generated. Specifically, the amount of first analyte contained in a test sample is inversely proportional to the first signal generated. Specifically, the amount of MPO or MPO fragment (mass) present can be quantified based on comparing the amount of light generated to a standard curve for the analyte or by comparison to a reference standard. The standard curve can be generated using serial dilutions or solutions of MPO or a MPO fragment of known concentration, by mass spectroscopy, gravimetrically and by other techniques known in the art.

After the MPO or the MPO fragment is detected and the amount of any MPO or (mass) quantified, the presence (or absence) of any autoantibodies to MPO or a MPO fragment is determined by performing a 3-dimensional (“3-D”) dose-response surface analysis of the data (also referred to as a “3-D standard ‘curve’”) based on combinations of the first analyte of interest and the second analyte of interest of known concentrations.

The use of dose-response surface analysis to identify significant variables in the optimization of assays, chemical reactions, etc. is the basis of design of experiments (“DOE”). Response surface analysis has also been used to study drug interactions (See, for example in, Civitico, G., Shaw, T., and Locarnini, S., Antimicrob Agents Chemother., 40, 1180-5 (1996)). Such analyses are generally qualitative analyses. In the present invention, such response surfaces are used for quantitative analysis. Any program known in the art can be used in performing the response surface analysis, such as TableCurve-3D (Systat Software, Inc., San Jose, Calif.). Such programs can be used to provide an automated surface-fitting, namely, the equation that best fits the contour of the surface, for quantitative analysis for use in the assays of the present invention.

### D. METHODS FOR DETERMINING THE AMOUNT OF MPO OR MPO FRAGMENTS IN A TEST SAMPLE

In another embodiment, the present invention relates to a method of determining the amount of MPO or MPO fragment (namely, MPO or MPO fragment mass) from a test sample. The determination of the amount of MPO or MPO fragment in a test sample can be used to assess the risk of a subject for cardiovascular disease (such as, but not limited to, myocarditis, ischemic heart disease, or hypertrophic or restrictive cardiomyopathy). More specifically, the methods of the present invention can be used to assess whether a subject is at risk of developing cardiovascular disease or is currently suffering from cardiovascular disease. In addition, the method of the present invention can also be used to assess the severity of a subject suffering from cystic fibrosis.

The assay or method of the present invention involves obtaining a test sample from a subject. A subject from which a test sample can be obtained is any vertebrate. Preferably, the vertebrate is a mammal. Examples of mammals include, but are not limited to, dogs, cats, rabbits, mice, rats, goats, sheep, cows, pigs, horses, non-human primates and humans. The test sample can be obtained from the subject using routine techniques known to those skilled in the art. Preferably, the test sample contains MPO or a MPO fragment.

The method also involves determining an amount of MPO activity in a test sample and the amount of MPO mass in the test sample. The determination of the amount of MPO activity in the test sample the amount of MPO mass in the test sample can be performed simultaneously or sequentially, in any order.

After the amount of MPO activity in the test sample and the amount of MPO mass in the test sample have been determined, then a comparison of these values is performed. The manner in which the comparison is made is not critical. For example, the values for each of the amount of MPO activity and the amount of MPO mass can be expressed as a ratio. The comparison can be made manually, such as by a human, or can completely or partially be performed by a computer program or algorithm, along with the necessary hardware, such as input, memory, processing display and output devices.

The value obtained for the amount of MPO activity in the test sample and the value obtained for the amount of MPO mass in the test sample should be equal (or if expressed as a ratio should be in the ratio of about 1:1) or, if not equal, should not vary by more than about ten percent (10%; e.g., from about 0.1% to about 10%), preferably, not more than about five percent (5%; e.g., from about 0.1% to about 5%).

After the above described comparison is made between the values for MPO activity and MPO mass, a determination is made as to the amount of MPO in the test sample.

### E. ASSAY KITS FOR DETERMINING THE RELIABILITY OF A MPO ASSAY RESULT FROM A TEST SAMPLE, FOR DETERMINING AUTOANTIBODIES TO MPO OR MPO FRAGMENT, AND FOR DETERMINING THE AMOUNT OF MPO OR MPO FRAGMENT IN A TEST SAMPLE

In another embodiment, the present invention relates to a test kit for determining the reliability of a MPO assay result from a test sample. The kit can contain one or more reagents for determining the amount of MPO activity in a test sample. The kit can also contain one or more reagents for determining the amount of MPO mass in a test sample. The kit can also contain one or more reagents for detecting autoantibodies to MPO or a MPO fragment in a test sample. Additionally, the kit can contain instructions for determining the reliability of a MPO assay result in a test sample. Such instructions optionally can be in a printed form or on CD, DVD, or other format of recorded media.

In yet another embodiment, the present invention relates to a test kit for detecting autoantibodies to MPO or a MPO fragment in a test sample. The kit can contain a first specific binding partner, wherein said first specific binding partner is MPO or a MPO fragment. Additionally, the kit can also contain a second specific binding partner wherein said second specific binding partner is an anti-human antibody. The kit can also contain at least one detectable label. The detectable label can be a separate component of the kit. Alternatively, the detectable label may be conjugated to the second specific binding partner and supplied in the kit in this form. Preferably, the detectable label is at least one acridinium compound. If the kit contains at least one acridinium compound, the acridinium compound may comprise at least one acridinium-9-carboxamide, at least one acridinium-9-carboxylate aryl ester or any combinations thereof. More specifically, the acridinium-9-carboxamide that can be used has the structure according to Formula I:

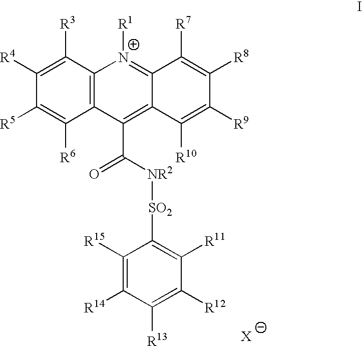

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

further wherein any of the alkyl, alkenyl, alkynyl, aryl or aralkyl may contain one or more heteroatoms; and

optionally, if present, X^{Θ} is an anion.

Additionally, the acridinium-9-carboxylate aryl ester that can be used has a structure according to formula II:

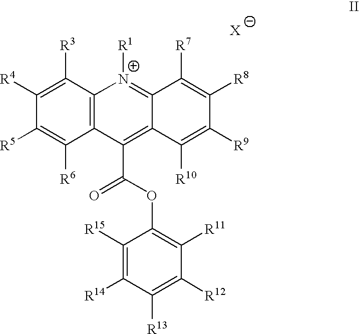

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{Θ} is an anion.

Additionally, the kit can also contain a means of generating hydrogen peroxide in situ in the test sample. A means for generating hydrogen peroxide in situ in the test sample can include adding at least one hydrogen peroxide generating enzyme. Alternatively, the kit can contain at least one source of hydrogen peroxide. The at least one source of hydrogen peroxide can be one or more buffers or other solutions that are known to contain hydrogen peroxide. Alternatively, the kit can contain a solution containing hydrogen peroxide.

Additionally, the kit can also contain at least one basic solution.

Additionally, the kit can also contain a solid phase. For example, the solid phase can be a magnetic particle, bead, test tube, microtiter plate, cuvette, membrane, a scaffolding molecule, film, filter paper, disc and chip.

Also, the kit can also contain one or more instructions for detecting and quantifying autoantibodies to MPO or a MPO fragment in a test sample. The kit can also contain instructions for generating a standard curve for the purposes of quantifying the autoantibodies or a reference standard for purposes of quantifying the autoantibodies in the test sample. Such instructions optionally can be in printed form or on CD, DVD, or other format of recorded media.

In another embodiment, the present invention provides a kit for detecting autoantibodies to MPO or a MPO fragment and MPO or a MPO fragment in a test sample. The kit can contain a source of hydrogen peroxide. Preferably, the source of hydrogen peroxide contains a predetermined concentration or amount of hydrogen peroxide. The source of hydrogen peroxide contained in the kit can be in the form of one or more buffers or other solutions that are known to contain hydrogen peroxide. Alternatively, the source of hydrogen peroxide can be a solution containing a predetermined concentration or amount of hydrogen peroxide.

The kit can also contain at least one acridinium compound, the acridinium compound may comprise at least one acridinium-9-carboxamide, at least one acridinium-9-carboxylate aryl ester or any combinations thereof. More specifically, the acridinium-9-carboxamide that can be used has the structure according to Formula I:

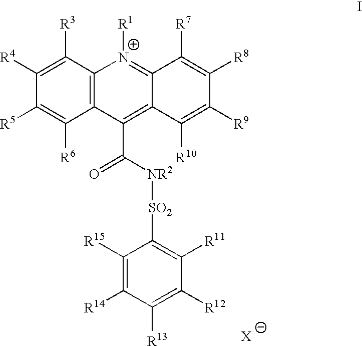

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

further wherein any of the alkyl, alkenyl, alkynyl, aryl or aralkyl may contain one or more heteroatoms; and

optionally, if present, X^{Θ} is an anion.

Additionally, the acridinium-9-carboxylate aryl ester that can be used has a structure according to formula II:

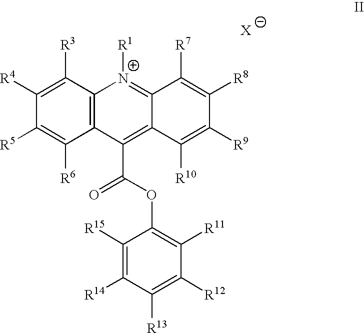

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{Θ} is an anion.

Additionally, the kit can also contain at least one basic solution.

Also, the kit can also contain one or more instructions for detecting and quantifying autoantibodies to MPO or a MPO fragment and MPO or a MPO fragment in a test sample. The kit can also contain instructions for generating a standard curve for the purposes of quantifying MPO or a MPO fragment or a reference standard for purposes of quantifying MPO or a MPO fragment in the test sample. Such instructions optionally can be in printed form or on CD, DVD, or other format of recorded media.

Also, the kit can also contain one or more instructions for performing three dimensional dose response surface analysis to calculate the amount of any MPO or MPO fragment and autoantibodies to MPO or a MPO fragment in a test sample. Such instructions optionally can be in printed form or on CD, DVD, or other format of recorded media.

In yet another embodiment, the present invention provides a kit for determining the amount of MPO or MPO fragment (namely mass) in a test sample. The kit can contain one or more reagents for determining the amount of MPO activity in a test sample. The kit can also contain one or more reagents for determining the amount of MPO mass in a test sample. Additionally, the kit can contain instructions for determining the amount of MPO or MPO fragment in a test sample. Such instructions optionally can be in a printed form or on CD, DVD, or other format of recorded media.

### F. ADAPTATIONS OF THE METHODS OF THE PRESENT INVENTION

The disclosure as described herein also can be adapted for use in a variety of automated and semi-automated systems (including those wherein the solid phase comprises a microparticle), as described, e.g., in U.S. Pat. Nos. 5,089,424 and 5,006,309, and as, e.g., commercially marketed by Abbott Laboratories (Abbott Park, IL) including but not limited to Abbott's ARCHITECT®, AxSYM, IMX, PRISM, and Quantum II instruments, as well as other platforms. Moreover, the disclosure optionally is adaptable for the Abbott Laboratories commercial Point of Care (I-STAT®) electrochemical immunoassay system for performing sandwich immunoassays. Immunosensors, and their methods of manufacture and operation in single-use test devices are described, for example in, U.S. Pat. No. 5,063,081, U.S. Patent Application 2003/0170881, U.S. Patent Application 2004/0018577, U.S. Patent Application 2005/0054078, and U.S. Patent Application 2006/0160164, which are incorporated in their entireties by reference for their teachings regarding same.

In particular, with regard to the adaptation of the present autoantibody assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized first specific binding partner (MPO or MPO fragment) are adhered to a polymer coating of patterned polyvinyl alcohol over the electrode. This chip is assembled into an I-STAT® cartridge with a fluidics format suitable for immunoassay. On a portion of the wall of the sample holding chamber of the cartridge there is a layer comprising the second MPO specific binding partner labeled with alkaline phosphatase (or other label). Within the fluid pouch of the cartridge is an aqueous reagent that includes p-aminophenol phosphate.

In operation, a sample suspected of containing MPO is added to the holding chamber of the MPO test cartridge and the cartridge is inserted into the I-STAT® reader. After the second specific binding partner has dissolved into the sample, a pump element within the cartridge forces the sample into a conduit containing the chip. Here it is oscillated to promote formation of the sandwich between the first specific binding partner, MPO and the labeled second specific binding partner. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of MPO in the sample by means of an embedded algorithm and factory-determined calibration curve.

It further goes without saying that the methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT® conjugate diluent employed in certain kits (Abbott Laboratories, Abbott Park, Ill.) and containing 2-(N-morpholino)ethanesulfonic acid (MES), other salt, protein blockers, antimicrobial and detetergent. An exemplary calibrator diluent is ARCHITECT® Human calibrator diluent employed in certain kits (Abbott Laboratories, Abbott Park, Ill.), which comprises a buffer containing MES, other salt, a protein blocker and an antimicrobial.

Furthermore, as previously mentioned, the methods and kits optionally are adapted for use on an automated or semi-automated system. Some of the differences between an automated or semi-automated system as compared to a non-automated system (e.g., ELISA) include the substrate to which the first specific binding partner (e.g., analyte antigen or capture antibody) is attached (which can impact sandwich formation and analyte reactivity), and the length and timing of the capture, detection and/or any optional wash steps. Whereas a non-automated format such as an ELISA may include a relatively longer incubation time with sample and capture reagent (e.g., about 2 hours) an automated or semi-automated format (e.g., ARCHITECT®) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT®) may have a relatively shorter incubation time (e.g., approximately 4 minutes for the ARCHITECT®).

One skilled in the art would readily appreciate that the present disclosure is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the disclosure. It will be readily apparent to one skilled in the art that varying substitutions and modifications may be made to the disclosure disclosed herein without departing from the scope and spirit of the disclosure.

All patents, patent application publications, journal articles, textbooks, and other publications mentioned in the specification are indicative of the level of skill of those in the art to which the invention pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

The terms and expressions, which have been employed, are used as terms of description and not of limitation. In this regard, where certain terms are defined under “Definitions” and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Definitions,” “Methods for Determining the Reliability of a MPO Assay Result from a Test Sample,” “Methods for Detecting Autoantibodies to MPO or a MPO Fragment in a Test Sample,” and “Methods for Detecting the Amount of MPO or a MPO Fragments in a Test Sample,” and “Assay Kits for Determining the Reliability of a MPO Assay Result from a Test Sample, for Determining Autoantibodies to MPO or a MPO Fragment, and for Determining the Amount of MPO or a MPO Fragments in a Test Sample” are used in the “Detailed Description,” such use is solely for ease of reference and is not intended to limit any disclosure made in one section to that section only; rather, any disclosure made under one subheading is intended to constitute a disclosure under each and every other subheading. For example, terms defined under “Definitions” constitute part of the disclosures made under the “Methods for Determining the Reliability of a MPO Assay Result from a Test Sample” and “Methods for Detecting Autoantibodies to MPO or a MPO Fragment in a Test Sample” subheadings, as well as other subheadings.

It is recognized that various modifications are possible within the scope of the claimed invention. Thus, it should be understood that, although the present invention has been specifically disclosed in the context of preferred embodiments and optional features, those skilled in the art may resort to modifications and variations of the concepts disclosed herein. Such modifications and variations are considered to be within the scope of the invention as defined by the appended claims.

What is claimed is:

1. A method of determining the reliability of a myeloperoxidase assay result from a test sample, the method comprising the steps of:
(a) providing a test sample;
(b) providing a myeloperoxidase assay result;
(c) determining an amount of myeloperoxidase activity in the test sample;
(d) determining an amount of myeloperoxidase mass in the test sample; and
(e) comparing the amount of myeloperoxidase activity determined in step (c) with the amount of myeloperoxidase mass determined in step (d) and using said comparison to determine the reliability of the myeloperoxidase assay result provided in step (b).

2. The method of claim 1, wherein the determining of step (c) and the determining of step (d) are done simultaneously.

3. The method of claim 1, wherein the determining of step (c) and the determining of step (d) are done sequentially, in any order.

4. The method of claim 1, wherein the test sample is whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid or semen.

5. The method of claim 1, wherein the myeloperoxidase activity is determined using an immunoassay or a chemiluminescent assay.

6. The method of claim 1, wherein the myeloperoxidase mass is determined using an immunoassay or a chemiluminescent assay.

7. The method of claim 1, wherein the myeloperoxidase assay result is determined using an immunoassay or a chemiluminescent assay.

8. A method for detecting autoantibodies to myeloperoxidase or a myeloperoxidase fragment in a test sample, the method comprising the steps of:
(a) preparing a mixture comprising a test sample being assessed for autoantibodies to myeloperoxidase or a myeloperoxidase fragment and a first specific binding partner that is immobilized on a solid phase, wherein the first specific binding partner is myeloperoxidase or a myeloperoxidase fragment and further wherein the autoantibody and the first specific binding partner form a solid phase first specific binding partner-autoantibody complex;
(b) removing any unbound autoantibodies from the solid phase first specific binding partner-autoantibody complex;
(c) adding a second specific binding partner labeled with a detectable label to the mixture to form a first specific binding partner-autoantibody-second specific binding partner complex, wherein the second specific binding partner is an anti-human antibody and the detectable label is an acridinium compound;
(d) removing any unbound second specific binding partner labeled with a detectable label from the first specific binding partner-autoantibody-second specific binding partner complex;
(e) generating in or providing to the mixture a source of hydrogen peroxide before or after the addition of the second specific binding partner containing the detectable label;
(f) adding a basic solution to the mixture to generate a light signal; and
(g) measuring the light generated to detect the autoantibody.

9. The method of claim 8, wherein the test sample is whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid or semen.

10. The method of claim 8, wherein the hydrogen peroxide is provided by adding a buffer or a solution containing hydrogen peroxide.

11. The method of claim 8, wherein the hydrogen peroxide is generated by adding a hydrogen peroxide generating enzyme to the test sample.

12. The method of claim 8, wherein the acridinium compound is an acridinium-9-carboxamide having a structure according to formula I: 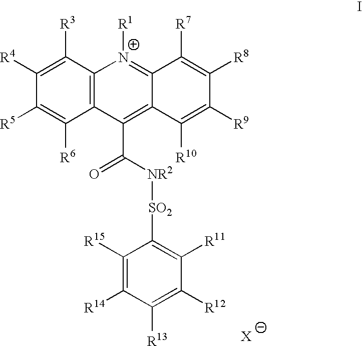
wherein R¹ and R² are each independently selected from the group consisting of: alkyl, alkenyl, alkynyl, aryl or aralkyl, sulfoalkyl and carboxyalkyl, and
wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, and carboxyalkyl; and
optionally, if present, X^{Θ} is an anion.

13. The method of claim 8, wherein the acridinium compound is an acridinium-9-carboxylate aryl ester having a structure according to formula II: 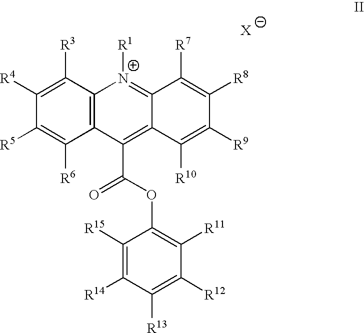
wherein R¹ is an alkyl, alkenyl, alkynyl, aryl or aralkyl, sulfoalkyl, carboxyalkyl and oxoalkyl; and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{Θ} is an anion.

14. The method of claim 8, further comprising quantifying the amount of autoantibodies to myeloperoxidase or a myeloperoxidase fragment in the test sample by relating the amount of light generated in the test sample by comparison to a standard curve for said autoantibodies.

15. The method of claim 14, wherein the standard curve is generated from solutions of autoantibodies of known concentrations.

16. An interdependent method for detecting autoantibodies to myeloperoxidase or a myeloperoxidase fragment and myeloperoxidase or a myeloperoxidase fragment in a test sample, the method comprising the steps of:
(a) adding a predetermined concentration of hydrogen peroxide to the test sample;
(b) adding an acridinium compound to the test sample before or after the addition of the hydrogen peroxide;
(c) adding a basic solution to the test sample to generate a light signal;
(d) measuring the light generated from the light signal and calculating the amount of myeloperoxidase or a myeloperoxidase fragment present in the test sample; and
(e) performing a three dimensional dose response surface analysis to calculate the amount of autoantibodies in the test sample.

17. The method of claim 16, wherein the test sample is whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid or semen.

18. The method of claim 16, further comprising quantifying the amount of myeloperoxidase or a myeloperoxidase fragment in the test sample by relating the amount of light generated in the test sample by comparison to a standard curve for myeloperoxidase or a myeloperoxidase fragment.

19. The method of claim 18, wherein the standard curve is generated from solutions of myeloperoxidase or a myeloperoxidase fragment of a known concentration.

20. The method of claim 16, wherein the acridinium compound is an acridinium-9-carboxamide having a structure according to formula I: 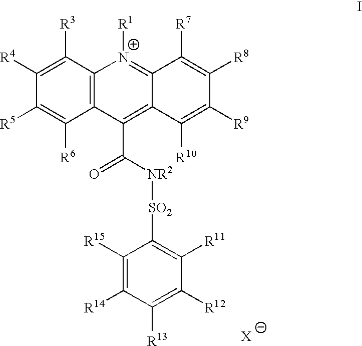
wherein R¹ and R² are each independently selected from the group consisting of: alkyl, alkenyl, alkynyl, aryl or aralkyl, sulfoalkyl and carboxyalkyl, and wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, and carboxyalkyl; and
optionally, if present, X^{Θ} is an anion.

21. The method of claim 16, wherein the acridinium compound is an acridinium-9-carboxylate aryl ester having a structure according to formula II: 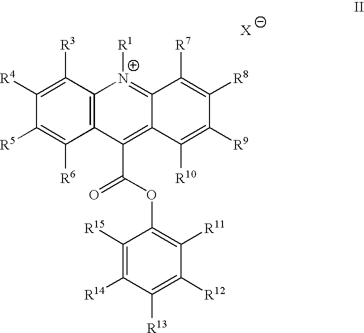
wherein R¹ is an alkyl, alkenyl, alkynyl, aryl or aralkyl, sulfoalkyl, carboxyalkyl and oxoalkyl; and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{Θ} is an anion.

22. A method for determining the amount of myeloperoxidase or a myeloperoxidase fragment in a sample, the method comprising the steps of:
(a) determining an amount of myeloperoxidase activity in the test sample;
(b) determining an amount of myeloperoxidase mass in the test sample; and
(c) comparing the amount of myeloperoxidase activity determined in step (a) with the amount of myeloperoxidase mass determined in step (b) and using said comparison to determine the amount of myeloperoxidase or myeloperoxidase fragment in the test sample.

23. The method of claim 22, wherein the determining of step (a) and the determining of step (b) are done simultaneously.

24. The method of claim 22, wherein the determining of step (a) and the determining of step (b) are done sequentially, in any order.

25. The method of claim 22, wherein the test sample is whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid or semen.

26. The method of claim 22, wherein the myeloperoxidase activity is determined using an immunoassay or a chemiluminescent assay.

27. The method of claim 22, wherein the myeloperoxidase mass is determined using an immunoassay or a chemiluminescent assay.

28. A test kit for determining the reliability of a myeloperoxidase assay result from a test sample, the kit comprising:
(a) one or more reagents for determining the amount of myeloperoxidase activity in the test sample;
(b) one or more reagents for determining the amount of myeloperoxidase mass in the test sample; and
(c) one or more reagents for detecting autoantibodies in the test sample.

29. The test kit of claim 28, wherein said test kit further comprises instructions for determining the reliability of a myeloperoxidase assay result from a test sample.

30. A test kit for detecting autoantibodies to myeloperoxidase or a myeloperoxidase fragment in a test sample, the kit comprising:
(a) a first specific binding partner, wherein said first specific binding partner is myeloperoxidase or a myeloperoxidase fragment; and
(b) a second specific binding partner, wherein said second specific binding partner is an anti-human antibody;
(c) at least one acridinium compound;
(d) at least one basic solution; and
(e) a source of hydrogen peroxide.

31. The test kit of claim 30, wherein said test kit further comprises instructions for determining the reliability of a myeloperoxidase assay result from a test sample.

32. The test kit of claim 30, wherein said test kit further comprises a solid phase.

33. The test kit of claim 30, wherein the acridinium compound is an acridinium-9-carboxamide having a structure according to formula I: 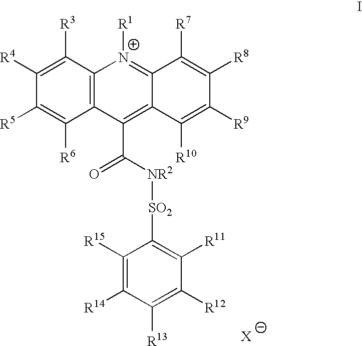
wherein R¹ and R² are each independently selected from the group consisting of: alkyl, alkenyl, alkynyl, aryl or aralkyl, sulfoalkyl and carboxyalkyl, and wherein R³ through R are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, and carboxyalkyl; and
optionally, if present, X^{Θ} is an anion.

34. The test kit of claim 30, wherein the acridinium compound is an acridinium-9-carboxylate aryl ester having a structure according to formula II: 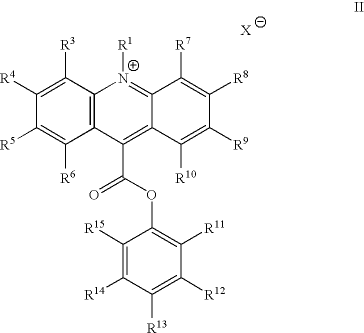
wherein R¹ is an alkyl, alkenyl, alkynyl, aryl or aralkyl, sulfoalkyl, carboxyalkyl and oxoalkyl; and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{Θ} is an anion.

35. A test kit for detecting autoantibodies to myeloperoxidase or a myeloperoxidase fragment and myeloperoxidase or a myeloperoxidase fragment in a test sample, the kit comprising:
(a) at least one acridinium compound;
(b) at least one basic solution; and
(c) a source of hydrogen peroxide, wherein said source contains a predetermined amount of hydrogen peroxide.

36. The test kit of claim 35, wherein said test kit further comprises instructions for performing a dimensional dose response surface analysis to calculate the amount of autoantibodies to myeloperoxidase or a myeloperoxidase fragment and myeloperoxidase or a myeloperoxidase fragment in the test sample.

37. The test kit of claim 35, wherein the acridinium compound is an acridinium-9-carboxamide having a structure according to formula I: 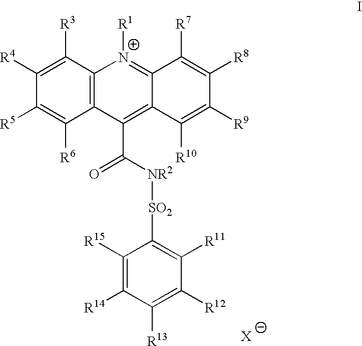
wherein R¹ and R² are each independently selected from the group consisting of: alkyl, alkenyl, alkynyl, aryl or aralkyl, sulfoalkyl and carboxyalkyl, and wherein R³ through R¹⁵ are each independently selected from the group consisting of :hydrogen; alkyl, alkenyl, alkynyl, aryl or aralkyl, amino, amido, acyl, alkoxyl, hydroxyl, carboxyl, halide, nitro, cyano, sulfo, sulfoalkyl, and carboxyalkyl; and
optionally, if present, X^{Θ} is an anion.

38. The test kit of claim 35, wherein the acridinium compound is an acridinium-9-carboxylate aryl ester having a structure according to formula II: 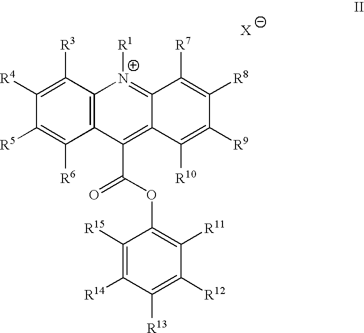
wherein R¹ is an alkyl, alkenyl, alkynyl, aryl or aralkyl, sulfoalkyl, carboxyalkyl and oxoalkyl; and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{Θ} is an anion.

39. A test kit for determining the amount of myeloperoxidase or a myeloperoxidase fragment in a test sample, the kit comprising:
(a) one or more reagents for determining the amount of myeloperoxidase activity in the test sample; and
(b) one or more reagents for determining the amount of myeloperoxidase mass in the test sample.

40. The test kit of claim 39, wherein said test kit further comprises instructions for determining the amount of myeloperoxidase or a myeloperoxidase fragment in a test sample.
